(19) United States
(12) Patent Application Publication
(10) Pub. No.: US 2005/0283122 A1
(43) Pub. Date: Dec. 22, 2005

(54) Slit valves bridging between the tip and distal side wall of catheter tubes and methods

(76) Inventor: Greg Nordgren, North Logan, UT (US)

Correspondence Address: Lynn G. Foster, 602 East 300 South, Salt Lake City, UT, 84102, US

(21) Appl. No.: 11/213,133

(22) Filed: Aug. 26, 2005

Related U.S. Application Data
(63) Continuation-in-part of application No. 10/647,039, filed on Aug. 21, 2003.
Application No. 10/647,039 is a continuation of application No. 09/542,503, filed on Apr. 3, 2000, now abandoned.

(51) Int. Cl. A61M 5/00

(52) U.S. Cl. 604/247

(57) ABSTRACT

Slit valves are disclosed which are placed in normally closed distal catheter tips so as to extend somewhat into the wall at the distal ends of catheter tubes whereby different rates of flow through the slit valves are achieved for aspiration and infusion, respectively.

### CONTINUITY

This application is a continuation-in-part of my co-pending U.S. patent application Ser. No. 10/647,039, filed Aug. 21, 2003, which is a continuation of Ser. No. 09/542,503, filed Apr. 3, 2000.

### FIELD OF INVENTION

The present invention relates generally to slit valves for catheters and, more particularly to a variety of configurations of slit valves disposed in otherwise closed catheter tips so as to also extend into the adjacent side wall of catheter tubes at the distal end thereof, and related methods.

### BACKGROUND

In the past, slit valves have traditionally been used only in the side walls of otherwise closed catheter tubes to infuse or aspirate fluid. Use of such side wall slit valves has been directed to infusion and aspiration of liquids in the cardiovascular systems of medical patients, infusion and aspiration of fluids in the respiratory systems of medical patients, and infusion and aspiration in other body cavities.

Disadvantageously, when a slit of a given slit valve, disposed in the catheter tube side wall, is open, sometimes a central passageway within the catheter tube is partially or totally occluded. Also, interference can occur between the indwelling lips forming a slit, as they are flexed outwardly, and the wall of the body cavity in which the catheter tube and slit valve are disposed, which either prevents the slit valve from opening or undesirably limits the extent to which it is permitted to open. Such side wall slit valves tend to flex open to the same extent for both infusion and aspiration.

### BRIEF SUMMARY AND OBJECTS OF THE INVENTION

In brief summary, the present invention overcomes or substantially alleviates past problems in the catheter-related slit valve field. One or more valves in a variety of forms are disposed in otherwise closed tips at the distal end of catheter tubes so as to extend somewhat into the adjacent catheter tube wall, whereby tube passageway occlusion problems and body cavity interference problems are greatly reduced, if not eliminated and influent and effluent liquid flow rate are different.

With the foregoing in mind, it is a primary object to overcome or substantially alleviate past problems in the catheter-related slit valve field.

Another paramount object is the provision of one or more slit valves in a variety of forms, in otherwise normally closed tips at the distal end of a catheter tube so as to extend somewhat into the adjacent side wall of the catheter tube.

A further dominate object is the provision of slit valves in the distal tips of catheter tubes so as to extend somewhat into the adjacent side wall of the catheter tube by which occlusion and interference problems are greatly reduced, if not eliminated.

Another valuable object is the provision of catheter tube slit valves whereby infusion and aspiration flow rates are different.

These and other objects and features of the present invention will be apparent from the detailed description taken with reference to accompanying drawings.

### BRIEF DESCRIPTION OF THE DRAWINGS

FIGS. 1 is a fragmentary perspective of one slit valve disposed primarily in a flat at a normally closed tip of a catheter tube, with the slit of the valve extending somewhat into the adjacent side wall of the catheter tube;

FIG. 2 is a cross section taken along lines 2-2, showing the slit valve flexed outwardly into an open aspirating condition;

FIG. 3 is a cross section similar to FIG. 2, but showing the slit valve flexed outwardly into a larger open infusing condition;

FIG. 4 is a fragmentary perspective of a second slit valve disposed, on a rounded portion, at a normally closed tapered tip of a catheter tube, with the slit of valve extending somewhat into the adjacent side wall of the catheter tube;

FIG. 5 is a cross section taken along lines 5-5 of FIG. 4;

FIG. 6 is a fragmentary perspective of a third embodiment showing a slit valve primarily disposed in a flat at a normally closed tip of a catheter tube, with the slit ending both across the apex of the tip and somewhat into the adjacent side wall of the catheter tube;

FIG. 7 is a cross section taken along lines 7-7 of FIG. 6;

FIG. 8 is a fragmentary perspective with parts broken away for clarity of a multi-lumen embodiment of the present invention, showing slit valves in accordance with the present invention for each lumen; and

FIG. 9 is a side elevation of the multi-lumen embodiment of FIG. 8.

### DETAILED DESCRIPTION OF THE ILLUSTRATED EMBODIMENTS

The present invention solves or reduces past problems in the catheter-related slit valve field, applicable to the human respiratory system, the human circulatory system, and other body cavities. Apart from whether a catheter tube comprises a sidewall slit valve or does not, one or more slit valves, which may be in a variety of forms, are disposed in an otherwise normally closed distal end of each catheter tube so as to extend somewhat into the wall of the catheter tube at the distal end thereof, whereby passageway occlusion problems and body cavity interference problems are greatly reduced, if not eliminated and the degree of flexure to open inwardly for aspiration is less than to open outwardly for infusion. In some embodiments a tip slit valve may traverse an apex of the tip.

Accordingly, the tip and the side wall at the distal end of a single or multiple lumen catheter tube may comprise one or more slit valves to both aspirate and infuse. One or more slit valves may be located in part in a rounded portion of the tip or in a flat region thereof, as deemed appropriate by those having skill in the art. If desired, the slit may traverse an apex of the tip. The distal tip may be configured as desired. For example, a bullet, a cone, a pyramid, hemispheric, angular, parabolic tip maybe used. See parent U.S. patent application Ser. No. 10/647,039. The slits in any of the slit valves, may have a uniform thickness or a thickness which varies, as deemed most appropriate for an intended purpose.

The preferred materials for forming the catheter tubes and the distal tips comprise silicone rubber, polyurethane and other suitable natural and synthetic elastomeric materials. The material at each slit valve must have sufficient flexibility for the lips forming the slit to flex inwardly or outwardly when predetermined pressure differentials are imposed thereon, in order to accommodate fluid flow in the direction desired. Treating the lips with a softening composition may take place to provide the desired flexibility.

Where multi-lumen catheter tubes are involved, typically the distal tip will comprise at least one slit valve to accommodate fluid flow into, out of, each lumen.

The slits may be parallel, perpendicular, staggered, radially disposed or otherwise oriented, as deemed most appropriate by those having skill in the art. Opposing or offset slit valves located on different sides of a tip may be used.

The present invention provides catheter assemblies which may be inserted into a medical patient over a guide wire. Pressures required for infusion and aspiration are lower than in the past. The distal tips and catheter tube walls may be formed in any suitable fashion.

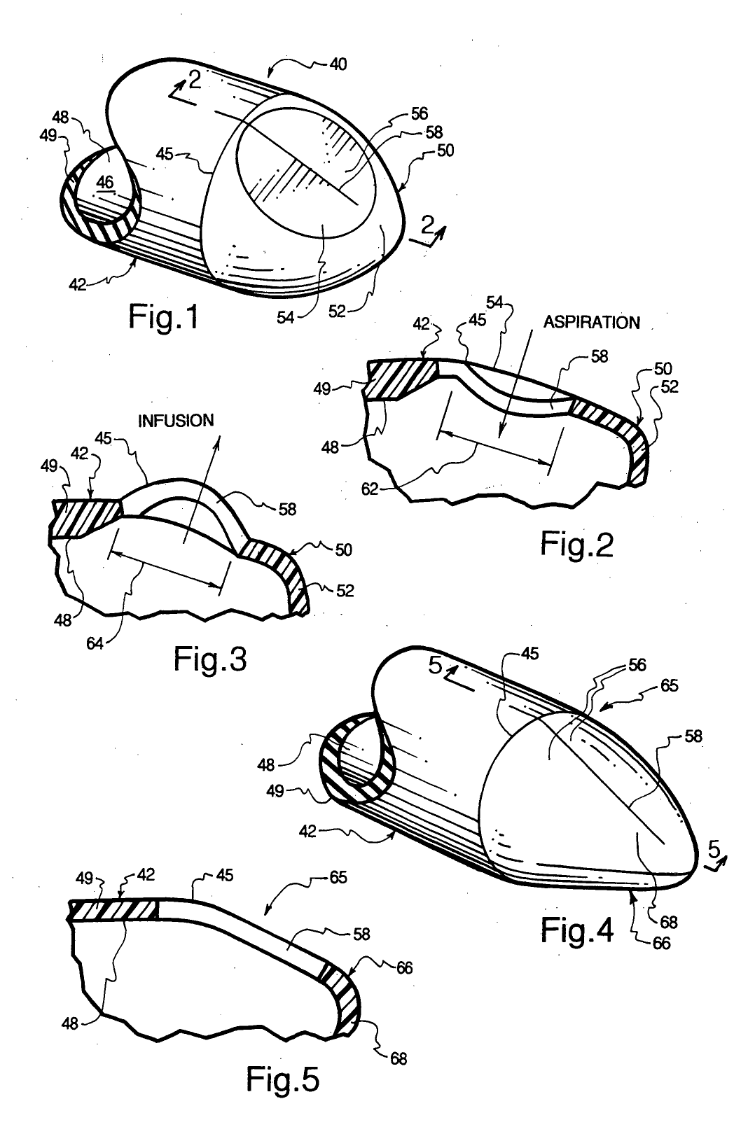

Reference is now made to the drawings wherein like numerals are used to designate like parts throughout. One or more slit valves located in a normally closed tip and extending somewhat into the side wall of a catheter tube may be used for infusing, aspirating or both and may be of any desired configuration. Each of the embodiments shown in FIGS. 1 through 9 comprise a normally closed tip at the distal end of a catheter tube assembly, each comprising a catheter tube, having a hollow interior forming one or more passageways for selective displacement of a fluid. Each passageway or lumen is defined within an annular wall which typically comprises an exterior annular surface. The distal end of each catheter tube is joined at an interface to a normally closed distal tip in which at least one slit valve is disposed so as to extend somewhat into the side wall of the adjacent catheter tube. The distal tip may be of any desired configuration. The catheter tube and distal tip may be formed as one piece or separately and thereafter integrated.

FIG. 1 illustrates a cone-shaped tip, generally designated 50, comprising wall 52. Tip 50 may be symmetrical or non-symmetrical and comprises a flat region 54 in which a slit valve 56 comprising 58 is predominantly disposed.

As shown in FIG. 1, catheter 40 comprises a catheter tube 42 comprising a single lumen 48 defined by an annular wall 49. The interior surface 46 is annular. The normally closed tip 50 comprises the wall 52, which is tapered and defines a flat region 54 in which the slit valve 56 is predominantly disposed. The slit 58 of the slit valve 56 extends beyond the tip into the wall 49 of the catheter tube 42.

As shown in FIGS. 2 and 3, the slit 58 extends somewhat into the wall 49 of the single lumen catheter tube 42, with the wall 52 of the tip being illustrated as being of less thickness than the wall 49 of the catheter tube 42. This configuration accommodates less inward flexure, due to pressure differential, for aspiration and greater outward flexure for infusion, as shown at dimensions 62 and 64 in FIGS. 2 and 3, respectively. Thus, the influent fluid flow rate into the catheter tube 42 through the flexed slit 58 is less than the effluent fluid flow rate.

The catheter 65 of FIGS. 4 and 5 is similar to the catheter of FIG. 1, except the slit valve 56 and the slit 58 thereof are predominantly disposed in a rounded portion 68 of a generally conically shaped tip 66. The catheter tube 42 comprises a single lumen 48 defined by an annular catheter tube wall 49, illustrated as having the same thickness as the wall defining the tip 66. See FIG. 5. The slit 58 of the slit valve 56 extends a short distance into the wall 49 of the catheter tube so as to achieve the same type of differential in aspirating and infusing fluid flow rates as described above, in conjunction with FIGS. 2 and 3.

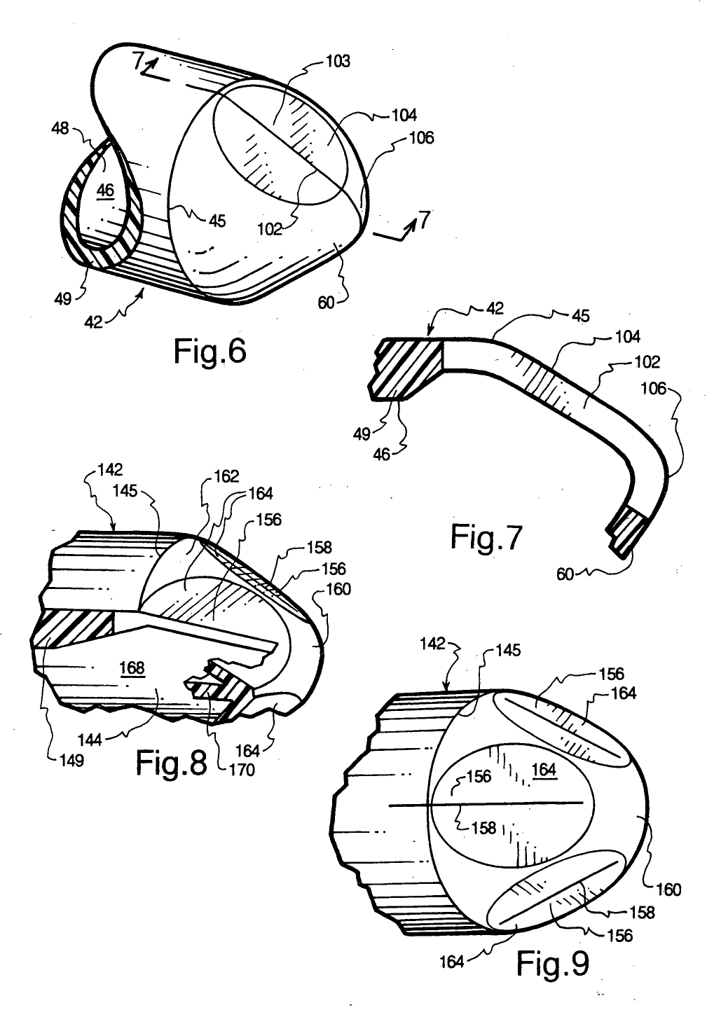

With reference to FIGS. 6 and 7, the distal tip 60 is shown to comprise an exterior flat surface 104 (aka flat 104) which is centrally bifurcated by a slit 102 comprising part of slit valve 103. The slit valve 103 is a two-way, aspirating and infusing valve, which opens when pre-determined thresholds of infusing and aspirating pressure differentials are imposed between the pressure inside the catheter tube 42 and the pressure outside the catheter tube 42 when indwelling within a body cavity of a medical patient. Thus, the infusion flow rate is greater than the aspiration flow rate. The slit 102 not only extends a short distance beyond the flat 104 into the wall 49 of the single lumen catheter tube 42 proximal of the interface 45 but also around the apex 106 of the tip 60.

FIGS. 8 and 9 illustrate a multi lumen catheter tube 142 wherein two slit valves 156 serve each of two lumens 144. Each slit valve 156 is illustrated as comprising a slit 158 extending from near the apex 16—of the normally closed tip 162 beyond interface 145 a short distance into the wall 149 of the catheter tube. The tip is strengthened or reinforced at the interior thereof by wall 170. The lumens 144 are completely separated from each other by a central divider 168.

Each slit 158 functions as described above in conjunction with FIGS. 2 and 3 to accommodate a larger opening, when flexed, for infusion than for aspiration. The multiple lumens can accommodate multiple infusions, including different medications at the same or different times, or simultaneous infusion and aspiration through the separate lumens.

The invention may be embodied in other specific forms without departing from the spirit of the central characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

1. A slit valve catheter comprising:
a catheter tube comprising at least one lumen;
a normally closed distal tip at the distal end of the catheter tube;
at least one normally closed slit valve comprising a slit disposed in the tip and extending into a wall of the catheter tube, the slit normally prohibiting fluid flow but opens when indwelling under a predetermined fluid pressure differential to accommodate fluid flow through the open slit.

2. A slit valve catheter according to claim 1 wherein the catheter tube comprises a plurality of internal lumens with at least one tip catheter tube wall slit valve associated with each lumen.

3. A slit valve catheter according to claim 1 wherein the at least one slit valve comprises a two way valve comprising lips adjacent to the slit which when indwelling selectively flex inwardly and outwardly depending on the pressure differential to aspirate and infuse to provide a larger flow rate for infusion than for aspiration.

4. A slit valve catheter according to claim 1 wherein the catheter tube is formed of a material selected from the group consisting of urethane, silicone rubber and natural and synthetic elastomers.

5. A slit valve catheter according to claim 1 wherein the tip is formed substantially of a material selected from the group consisting of urethane, silicone rubber and natural and synthetic elastomers.

6. A slit valve catheter according to claim 1 wherein the slit valve is formed of a material selected from the group consisting of urethane, silicone rubber and natural or synthetic elastomeric material treated to enhance flexure from the normally closed to an open position.

7. A slit valve catheter according to claim 1 wherein the catheter tube and the tip are formed as one piece.

8. A slit valve catheter according to claim 1 wherein the catheter tube and tip are first formed as two pieces and then integrated.

9. A combination comprising a catheter tube comprising a wall and a normally closed distal catheter tip comprising a tip wall and at least one normally closed but selectively openable slit valve extending through both the wall of the catheter tube and the tip wall.

10. A combination according to claim 9 wherein the tip configuration is selected from the group consisting of symmetric or asymmetric tips.

11. A combination according to claim 9 wherein the slit of the slit valve varies in thickness along its length.

12. A combination according to claim 9 wherein the tips are internally reinforced.

13. A combination according to claim 9 wherein the catheter is multi-lumen and at least one slit valve is associated with each lumen.

14. A combination according to claim 9 wherein the slit of the slit valve traverses around an apex of the tip.

15. A combination according to claim 9 wherein the tip comprises a wall thickness less than the catheter tube wall thickness.

16. A combination according to claim 9 wherein the slit valve comprises a two-way valve which flexes inwardly and outwardly responsive to pressure differential to create a larger infusion flow opening and a smaller aspiration flow opening.

17. A method of using a normally closed catheter tube comprising a distal normally closed tip having at least one normally closed slit valve bridging between the tip and the catheter tube, comprising the acts of:
placing the tip indwelling in a patient;
creating a fluid pressure differential across the normally closed slit valve such that the slit valve flexes inwardly and outwardly to open positions to accommodate a greater infusion fluid flow and a lesser aspiration fluid flow through the slit valve.
